(19) United States
(12) Patent Application Publication
(10) Pub. No.: US 2021/0196737 A1
(43) Pub. Date: Jul. 1, 2021

(54) USE OF TREZASTILBENOSIDE IN MANUFACTURE OF PRODUCT FOR TREATING AND/OR PREVENTING NON-ALCOHOLIC FATTY LIVER DISEASE

(71) Applicant: KPC PHARMACEUTICALS, INC., Kunming (CN)

(72) Inventors: Yidan LIU, Kunming (CN); Junfeng LIU, Kunming (CN); Xujuan YANG, Kunming (CN); Xi HUANG, Kunming (CN); Qing HUANG, Kunming (CN); Ke MENG, Kunming (CN); Guoguang LIU, Kunming (CN); Jianhua SHANG, Kunming (CN)

(73) Assignee: KPC PHARMACEUTICALS, INC., Kunming (CN)

(21) Appl. No.: 17/137,311

(22) Filed: Dec. 29, 2020

(30) Foreign Application Priority Data
Dec. 30, 2019 (CN) 201911393441.1

(51) Int. Cl. A61K 31/7034; A61P 3/06; A61P 1/16; A61K 9/20; A61K 9/48; A61K 9/16

(52) U.S. Cl. CPC A61K 31/7034; A61P 3/06; A61P 1/16; A61K 9/2054; A61K 9/2059; A61K 9/2027; A61K 9/2031; A61K 9/4858; A61K 9/4866; A61K 9/1623; A61K 9/1617; A61K 9/1652; A61K 9/2009

(57) ABSTRACT

The invention belongs to the technical field of biopharmaceuticals, and discloses the application of Trezastilbenoside in the manufacture of products for treating and/or preventing non-alcoholic fatty liver disease. The research of the present invention shows that after giving Trezastilbenoside to non-alcoholic fatty liver disease (NAFLD) model mice for 4 consecutive weeks, the serum TC, TG, and LDL content were all significantly reduced, and the HDL content was significantly increased, indicating that the drug has a lipid regulatory effect. The activities of AST and ALT in the serum were significantly weakened, and the infiltration of inflammatory factors in the liver tissue was reduced, indicating that the drug has a hepatoprotective effect. It can not only reduce the fat content of the liver, but also improve the pathological form of fatty liver, indicating an effect of against NASH.

### CROSS REFERENCE TO RELATED APPLICATION

The present application claims priority to Chinese Patent Application No. 201911393441.1, titled “USE OF TREZASTILBENOSIDE IN MANUFACTURE OF PRODUCT FOR TREATING AND/OR PREVENTING NON-ALCOHOLIC FATTY LIVER DISEASE”, filed on Dec. 30, 2019 with the Chinese Patent Office, which is incorporated herein by reference in its entirety.

### FIELD

The present disclosure belongs to the technical field of medicine, and specifically relates to the application of Trezastilbenoside in the manufacture of a product for treating and/or preventing non-alcoholic fatty liver disease.

### BACKGROUND

Non-alcoholic fatty liver disease (NAFLD) refers to a metabolic disease of the liver that is caused by long-term heavy drinking and other clear liver damage factors, and the accumulation of lipids mainly composed of triglycerides in liver cells is a pathological change. In NAFLD patients, liver fat metabolism is impaired, which causes a large amount of fatty substances to accumulate in liver cells (simple fatty liver), which in turn leads to liver cell steatosis, liver cell damage, inflammation, and liver fibrosis (non-alcoholic steatohepatitis), NASH). Simple fatty liver is a relatively benign stage of NAFLD, which is easily reversed. 10%-20% of simple fatty liver can progress to NASH. It is currently believed that NASH is an important link in the progression of NAFLD to end-stage liver disease such as liver cirrhosis, hepatocellular carcinoma, liver failure, and may become the primary cause of liver transplantation in the future. NAFLD has become one of the common liver diseases in clinical practice. Epidemiological investigations show that the incidence of NAFLD in our country is about 15%, while the incidence of NAFLD in Europe and the United States is more than 20%. Therefore, it is of great significance to explore effective treatments for NAFLD.

NAFLD pathologically generally includes three types: simple fatty liver, and steatohepatitis (NASH) and cirrhosis that evolved from simple fatty liver. Although hepatitis C, autoimmune liver disease, Wilson's disease, etc. can also cause liver steatosis, the main body of the disease is in the portal area and has a specific name, it does not belong to the category of ordinary fatty liver disease. The pathogenesis of fatty liver is not yet fully understood. It is currently believed that the “second hit” theory may be the common pathogenesis of alcoholic fatty liver and non-alcoholic fatty liver. Alcohol, obesity, diabetes, etc., as the first hit, caused the imbalance between triglyceride synthesis and metabolism in liver cells to cause fat storage to form simple fatty liver; the second hit refers to the action of the lipid peroxidation and inflammatory cytokines related to oxygen stress, leading to inflammation, necrosis and fibrosis of fatty liver cells. The difference is that alcoholic fatty liver is mainly caused by ethanol and its metabolites, while non-alcoholic fatty liver is mainly related to insulin resistance (Fan Jiangao, alcoholic and non-alcoholic fatty liver disease, Chinese Journal of Hepatology, 2003, 11(11):692). In NAFLD patients, lipid metabolism disorders are more common. The liver plays a major role in the process of lipid metabolism in the body. It can take in free fatty acids, process, store and export lipids. Problems in any part of the process may lead to the production of NAFLD. Free fatty acids play an important role in cells, such as synthesizing cell membranes, acting as energy storage, and participating in intracellular signaling pathways. However, chronic increases in free fatty acid content in many organs can disrupt metabolic pathways and induce insulin resistance (IR). The accumulation of lipids in the liver is closely related to the limit. Adipose tissue insulin resistance can increase lipolysis and increase the input of free fatty acids from adipose tissue to the liver, reducing output. In addition, the increase of content of reactive oxygen species (reactive oxygen species, ROS) in the body and a variety of cytokines (such as tumor necrosis factor α (TNF-α) and interleukin-6 (IL-6), etc.) may also lead to abnormal lipid metabolism of liver cells, leading to the occurrence or aggravation of NAFLD.

At present, there is no specific and effective treatment for NAFLD. Clinically used drugs for the treatment of fatty liver mainly include three categories: first, drugs for the primary disease, for example, obese patients can use Xenical, sibutramine, etc. to lose weight; patients with type 2 diabetes can use metformin, troglitazone, etc.; Second, liver-protecting drugs, which can use antioxidants, anti-inflammatory choleretics, liver-protecting and enzyme-lowering Chinese medicines, etc.; Third, lipid-lowering drugs, which improve liver fat deposition by reducing plasma lipid content. However, the efficacy of the above three types of drugs is uncertain, and some drugs have problems such as hepatotoxicity and multiple side effects. In addition, some of the lipid-lowering drugs that promote the transport of lipids in the blood to the liver for metabolism in the conventional treatment of fatty liver can reduce blood lipids, and at the same time they may increase liver fat and increase liver fat deposition. Several drugs have been used in clinical trials of NAFLD and NASH, but due to inconsistent outcomes and/or lack of therapeutic benefits in randomized controlled trials, they have not been recommended. Therefore, it is particularly important to develop effective drugs for intervention to prevent the progression of NAFLD disease.

Trezastilbenoside ((E)-1-(3,5-dihydroxyphenyl)-2-(3-hydroxy-4-O-(3-D-glucopyranosephenyl)ethylene or 3,5,3′,4′-trihydroxystibene-3′-O-β-glucoside) has a plant source of the rhizome of Rheum lhasaense A.J.Li et P.K.Hsiao. As suggested from the safety research, Trezastilbenoside had good safety, since no toxic side reactions and death occurred in acute toxicity tests, and no chromosome aberrations or mutagenesis were found in the genetic test. The structural formula of Trezastilbenoside is shown in Formula I:

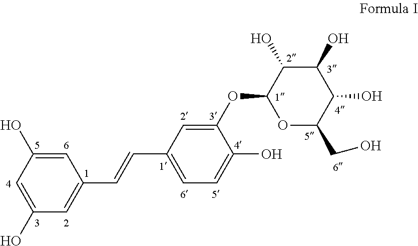 [SMILES: OC[C@H]1O[C@@H](Oc2cc(/C=C/c3cc(O)cc(O)c3)ccc2O)[C@H](O)[C@@H](O)[C@@H]1O]

Chinese Patent Application No. 201010116358.2 discloses use of Trezastilbenoside in preparation of a formulation for treating and preventing cardio-cerebro ischemia diseases and preparation method thereof. Chinese Patent Application No. 2011110371198.0 discloses a high performance liquid chromatograph method for measuring Trezastilbenoside content in Rheum lhasaense A.J.Li et P.K.Hsiao, in which an extraction process and a detection method for Trezastilbenoside are disclosed. The prior art shows that Trezastilbenoside has activities of treating ischemia cardio-cerebrovascular disease, but so far there is no report on its use for treating and preventing NAFLD.

In view of that, the present disclosure is provided.

### SUMMARY

In order to solve the technical problems and overcome the disadvantage of the prior art, the present disclosure provides use of Trezastilbenoside in manufacture of a product for treating and/or preventing non-alcoholic fatty liver disease.

The technical solutions with the following basic concepts are adopted by the present disclosure, so as to solve the technical problem described above.

The first object of the present disclosure is to provide use of Trezastilbenoside in manufacture of a product for treating and/or preventing non-alcoholic fatty liver disease.

Said Trezastilbenoside in the present disclosure may be a Trezastilbenoside extract, or the pure Trezastilbenoside compound. The preparation method of Trezastilbenoside may refer to the Chinese Patent Application No. 201010116358.2.

The research of the present invention shows that after giving the non-alcoholic fatty liver disease (NAFLD) model mice Trezastilbenoside for 4 consecutive weeks, the serum TC, TG, and LDL content were all significantly reduced, and the HDL content was significantly increased, indicating that the drug has a lipid-regulatory effect. The activity of AST and ALT in the serum were significantly weakened, and the infiltration of inflammatory factors in the liver tissue was reduced, indicating that the drug has a hepatoprotective effect; it can not only reduce the fat content of the liver, but also improve the pathological form of fatty liver, indicating a confronting effect against NASH. The results of this test suggest that Trezastilbenoside has good development and application value in the prevention and treatment of non-alcoholic fatty liver disease.

In a further embodiment, the non-alcoholic fatty liver disease includes simple fatty liver, steatohepatitis and hepatic sclerosis.

The second object of the present disclosure is to provide use of Trezastilbenoside in manufacture of a product for treating and/or preventing an increase in body weight and/or liver weight in a subject with non-alcoholic fatty liver disease.

The third object of the present disclosure is to provide use of Trezastilbenoside in manufacture of a product for treating and/or preventing an increase in total cholesterol and/or triglycerides in the serum of a subject with non-alcoholic fatty liver disease.

Specially, use of Trezastilbenoside in manufacture of a product for treating and/or preventing an increase in total cholesterol and/or triglycerides in the liver of a subject with non-alcoholic fatty liver disease, is provided.

The fourth object of the present disclosure is to provide use of Trezastilbenoside in manufacture of a product for treating and/or preventing an increase in aspartate aminotransferase and/or alanine aminotransferase in the serum of a subject with non-alcoholic fatty liver disease.

The fifth object of the present disclosure is to provide use of Trezastilbenoside in manufacture of a product for treating and/or preventing an increase in low density lipoprotein in the serum of a subject with non-alcoholic fatty liver disease.

The sixth object of the present disclosure is to provide use of Trezastilbenoside in manufacture of a product for treating and/or preventing a decrease in high density lipoprotein in the serum of a subject with non-alcoholic fatty liver disease.

In a further embodiment, the product includes a food product, a health product and a medicament.

In a further embodiment, the product for treating and/or preventing non-alcoholic fatty liver disease is a medicament, comprising Trezastilbenoside and a pharmaceutically acceptable adjuvant.

In a further embodiment, the medicament for treating and/or preventing non-alcoholic fatty liver disease is provided as various acceptable dosage forms.

Preferably, the dosage forms include injections, tablets, capsules, powders, pills or oral liquids.

The seventh object of the present disclosure is to provide a product for treating and/or preventing non-alcoholic fatty liver disease, wherein the product comprises an active ingredient of Trezastilbenoside.

In this embodiment, the product for treating and/or preventing non-alcoholic fatty liver disease comprises active ingredients containing Trezastilbenoside, and may be in form of a food product, a health product or a medicament.

Preferably, the product is a medicament, comprising Trezastilbenoside and a pharmaceutically acceptable adjuvant.

By adopting the technical solutions described above, the present disclosure produces the following beneficial effect as compared to the prior art.

The present invention discloses a new medicinal use of Trezastilbenoside. Through in vivo and in vitro studies, the present invention proves that Trezastilbenoside has remarkable effects on treating fatty liver and improving fatty degeneration of cultured liver cells; it can significantly reduce liver index and the content of serum TG and liver tissue TG, while inhibiting lipid peroxidation, significantly improves liver cell steatosis, and has important clinical application value for the prevention and treatment of fatty liver. The research results can develop the effective ingredient products of modern Chinese medicine for the treatment of NAFLD, and lay a good foundation for the development of modern Chinese patent medicine for the treatment of NAFLD with independent intellectual property rights. The effective ingredient of the present invention is easy to separate and extract from plants, can be used in industrial production, have a positive effect, and have a good market prospect.

The specific embodiments of the present invention will be described in further detail below in conjunction with the accompanying drawings.

### BRIEF DESCRIPTION OF DRAWINGS

The accompanying drawings are used as a part of the present disclosure to provide a further understanding of the present disclosure. The exemplary embodiments and descriptions of the present disclosure are used to explain the present disclosure, but do not constitute an improper limitation of the present disclosure. Apparently, the drawings in the following description are only some embodiments. For those of ordinary skill in the art, other drawings can be obtained based on these drawings without inventive work. In the drawings:

FIG. 1 is an image showing the pathological changes in mouse liver under a microscope (HE, ×200) of the present disclosure,

wherein A is the normal control group, B is the model group, C is the Trezastilbenoside low dose group, and D is the Trezastilbenoside high dose group.

It should to be noted that these drawings and the word description are provided to explain the concept of the present disclosure by referring to particular examples for these skilled in the art, but are not intended to limt the scope of the concept of the present disclosure in any way.

### DETAILED DESCRIPTION

In order to make the purposes, technical solutions and advantages of examples of the present disclosure clearer, hereafter the technical solutions in the examples will be described clearly and completely in conjunction with the drawings. The following examples are given to describe the present disclosure, and are not intended to limit the scope of the present disclosure.

### Example 1: The Protective Effect of Trezastilbenoside on Non-Alcoholic Hepatocyte Injury

### 1. Experimental Materials

### 1.1 Testing Samples

Trezastilbenoside, with a molecular weight of 406, white crystalline or crystalline powder, 99.6% purity and a lot number of 20120402.

### 1.2 Experiment Animals

44 healthy male C57BL/6 mice with a body weight of 19-23 g, purchased from Hunan SJA Laboratory Animal Co., Ltd with Laboratory Animal Production License No. SCXK(XIANG)2016-0002, were housed in groups inside PVC transparent plastic boxes of 6 mice or less in each box, at Research Institute of KPC Pharmaceuticals, Inc. The mice were given corresponding feed every day and received water ad libitum, with the cages and bedding changed when needed, at a temperature of 20-25° C. (daily temperature difference was ≤3° C.), a humidity of 40%-70%, under the illumination in a mode of 12 h: 12 h light/dark with an illumination intensity of 150-300 lx, a noise of 60 dB or less, and with a Laboratory Animal Use Permit NO. SYCK (DIAN) K2019-0001 issued by the Kunming Science and Technology Bureau.

### 1.3 Feed

A high-sugar and high-fat feed D09100310 containing 40% of fat, 20% of fructose and 2% of cholesterol, from Shuyishuer Bio-Technology Co., Ltd, was irradiated at 15 kGy;

An ordinary feed was from Jiangsu Xietong Medical Bioengineering Co., Ltd., with a permit No. Susizheng (2014) 01008.

### 1.4 Reagents and Instruments

Paraformaldehyde (analytical grade, Sinopharm Chemical Reagent Co., Ltd.), disodium hydrogen phosphate (analytical grade, Tianjin Fengchuan Chemical Reagent Technology Co., Ltd.), and sodium dihydrogen phosphate (analytical grade, Tianjin Fengchuan Chemical Reagent Technology Co., Ltd.) were employed. Reagent kits such as alanine aminotransferase (ALT), aspartate aminotransferase (AST), triglycerides (TG), total cholesterol (TC), low density lipoprotein (LDL) and high density lipoprotein (HDL) were all original reagents produced by Nanjing Jiancheng Bioengineering Institute. Xylene, absolute ethanol, formaldehyde, acetone, hematoxylin, Sudan IV, eosin, hydrochloric acid, ethanol, neutral gum, glycerin, gelatin, etc. were reagents required by pathological testing. Picric acid, sodium chloride injection solution, disinfection alcohol, etc. were common reagents used in laboratories. 96-well flat-bottom cell culture plates, embedding cassettes, paraffin wax, glass slides, cover glasses, slicing knives, etc. were used.

A tissue homogenizer, a high-speed freezing centrifuge, an microplate reader, an electronic scale, and an analytical balance; a dewaterer, LEICA ASP300S; a freezing microtome, LEICA CM1950; an embedding machine, LEICA EG1150H; a freezing table, LEICA EG1130; a rotary section machine, LEICA RM2235; a dryer, LEICA HI1220, etc. were employed.

### 2. Test Methods

### 2.1 Preparation of Animal Models

After 1 week of adaptive feeding, 44 C57 mice were divided into a control group (12 mice) and a model group (32 mice) according to the random number method. The normal control group was given ordinary diet, and the model group was given high-sugar and high-fat diet. After feeding for 8 weeks, randomly select 2 animals in each of the control group and the model group. After pathological examination confirmed that the modeling was successful, the model animals were divided into groups.

### 2.2 Grouping and Administration

The model animals were divided according to random number method into 3 groups of 10 mice each, namely model control group (equal volume of sodium chloride injection solution), Trezastilbenoside high dose group (20 mg/kg) and Trezastilbenoside low dose group (10 mg/kg). The mice in each administration group were administered intragastrically with the corresponding drug, once a day continuously for 4 weeks, while being fed with the high-sugar and high-fat feed continuously. The mice in the normal control group (equal volume of sodium chloride injection solution) were feed with the ordinary feed.

### 2.3 Observation Indicators

### 2.3.1 General Observation

After the last administration, the mice were fasted but had access to water for 12 h, and then sacrificed after blood was taken. The body mass and liver mass of each mouse were weighed to calculate the liver index by the following formula.

liver index=(liver wet mass/body mass)×100% (wet liver mass/body mass).

### 2.3.2 Measurement of the Levels of Serum TC, TG, HDL, LDL, AST and ALT

About 1 mL of blood, taken via mouse eyeballs, was placed into a 1.5 mL centrifuge tube, and then centrifuged at 3000 r/min for 10 min to obtain the supernatant. The levels of TC, TG, HDL, LDL, AST and ALT in the serum were measured with a microplate reader strictly under the operating instructions of the assay kit.

### 2.3.3 Measurement of TC and TG Content in Liver Tissue

About 100 mg of liver tissue weighed, was put into 900 μL absolute ethanol to make a 10% tissue homogenate under an ice bath. Then the supernatant was separated out through a centrifugation separation at 3000 r/min for 15 min, and collected into a 1.5 mL centrifuge tube for the measurement of TC and TG content in the liver tissue.

### 2.4.4 HE Staining of Liver

The fresh liver tissue was immersed and fixed in 4% formaldehyde solution overnight for 24 h, dehydrated through 75%-95%-100% ethanol gradient, embedded in paraffin, sectioned into 4 μm, and conventionally stained with hematoxylin-eosin, to observe the pathological changes of the liver tissue under an optical microscope (HE, 200×).

### 3. Statistical Methods

Experiment data were statistically analyzed using SPSS 20.0 software. The measurement data were expressed as X̅±s. T test was used for comparison between groups. A P-value less than 0.05 means that the difference is statistically significant.

### 4. Experiment Results

### 4.1 Effects on the Body Weight, Wet Liver Weight and Liver Index as Well as the General Conditions of Mice

During the experiment, the mice in each group were in good condition and had a strong appetite. Among them, the mice in the normal group showed a more docile temperament, with smooth fur and normal activities; the mice in the NAFLD model group gained rapid weight, and showed more irritable temperament and did not like to move. After 12 weeks of high-sugar and high-fat feeding, compared with the normal control group (normal diet), the weight gain, liver wet weight and liver index of the model group (high-sugar and high-fat diet) were significantly higher than those of the normal control group (P<0.01); Compared with the model group, the Trezastilbenoside administration group has a tendency to reduce the obesity induced by high sugar and high fat in mice, and significantly reduced the wet weight and liver index of the model animals (P<0.05). The results are shown in the table 1.

TABLE 1
Comparisons of the body weight, liver weight and liver index of mice ( [symbol] , g)

| Groups | N | Weight (g) | Wet liver weight (g) | Liver index (%) |
|---|---|---|---|---|
| Normal group | 10 | 25.7 ± 0.63 | 0.99 ± 0.22 | 0.38 ± 0.02 |
| Model group | 10 | 36.2 ± 0.55^{##} | 2.14 ± 0.21^{##} | 0.59 ± 0.14^{##} |
| Trezastilbenoside low dose group | 10 | 35.0 ± 0.62 | 1.84 ± 3.0* | 0.53 ± 0.0.4* |
| Trezastilbenoside high dose group | 10 | 33.8 ± 0.74 | 1.72 ± 2.7* | 0.51 ± 0.05* |

Note:
^{##}P-value less than 0.01 as compared to the normal control group;
*P-value less than 0.05 as compared to the model group

### 4.2 Comparisons of the Levels of TC, TG, HDL, LDL, AST and ALT in Serum

Compared with the normal control group, the serum levels of TC, TG, LDL and the activities of AST and ALT in the model group were significantly increased, and the HDL content was significantly reduced (P<0.05/0.01); compared with the model group, in the high-dose Trezastilbenoside group, the contents of TC, TG, LDL in the serum of mice were significantly reduced, the activities of AST and ALT were significantly weakened, and the content of HDL was significantly increased (P<0.01), indicating that Trezastilbenoside has a good protective effect on non-alcoholic fatty liver disease induced by high sugar and high fat. The results are shown in Table 2.

TABLE 2
Comparisons of the changes of TC, TG, HDL, LDL, AST
and ALT in the mouse serum (x̅ ± s, n = 10)

| | TC | TG | HDL | LDL | AST | ALT |
|---|---|---|---|---|---|---|
| Groups | (mmol/L) | (mmol/L) | (mmol/L) | (mmol/L) | (U/L) | (U/L) |
| Normal group | 2.63 ± 0.27 | 0.77 ± 0.08 | 1.53 ± 0.14 | 0.49 ± 0.21 | 143.56 ± 61.80 | 97.86 ± 8.51 |
| Model group | 6.89 ± 0.67^{#} | 2.80 ± 0.21^{#} | 0.09 ± 0.50^{#} | 0.67 ± 0.30^{#} | 232.22 ± 46.90^{#} | 165.24 ± 10.52^{##} |
| Trezastilbenoside | 5.53 ± 0.52* | 2.67 ± 0.19 | 0.27 ± 0.67 | 0.47 ± 0.13* | 147.73 ± 52.98* | 122.45 ± 27.37* |
| low dose group | | | | | | |
| Trezastilbenoside | 3.43 ± 0.17** | 0.99 ± 0.11** | 0.45 ± 0.16** | 0.41 ± 0.30* | 118.92 ± 10.75** | 78.30 ± 11.53** |
| high dose group | | | | | | |

Note:
^{#}P-value less than 0.05,
^{##}P-value less than 0.01, as compared to the normal control group;
*P-value less than 0.05,
**P-value less than 0.01, as compared to the model group.

### 4.3 Effects on the TC and TG Content in the Mouse Liver Tissue

Changes in liver fat content can better reflect the degree of fatty liver, and should be taken as one of the important indicators for judging the efficacy of anti-fatty liver drugs. After the mice were given the high-sugar and high-fat feed for 12 weeks, their liver TG and TC levels were all significantly increased. Trezastilbenoside can significantly reduce the liver TG and TC levels of fatty liver mice, which are statistically different from the model control group (P<0.05/0.01), the results are shown in Table 3.

TABLE 3
Effects on the lipid content in the mouse liver ( [symbol] , mg/g)

| Groups | TC (mg/g) | TG (mg/g) |
|---|---|---|
| Normal group | 3.08 ± 0.54 | 1.34 ± 0.18 |
| Model group | 7.22 ± 0.85^{##} | 3.25 ± 0.81^{##} |
| Trezastilbenoside low dose group | 6.53 ± 0.59* | 2.12 ± 0.58 |
| Trezastilbenoside high dose group | 4.23 ± 0.87** | 1.96 ± 0.99** |

Note:
^{##}P-value less than 0.01, as compared to the normal control group;
*P-value less than 0.05,
**P-value less than 0.01, as compared to the model group.

### 4.4 Gross Observation of Mouse Liver and Pathological Observation of Liver Tissue

### 4.4.1 Gross Observation of Mouse Liver

The liver of the mice in the normal control group was dark red, bright in color, soft and fragile. The liver was slightly wedge-shaped, the right end was round and thick, and the left end was narrow and thin without greasy feeling; the mouse liver in the model group was enlarged and yellow in color; the texture was hard, with granular and greasy feeling when pinched; the liver color of the mice in the low and high dose groups of Trezastilbenoside was between the normal control group and the model group, and the texture and shape tended to be normal.

### 4.4.2 HE Observation of Mouse Liver

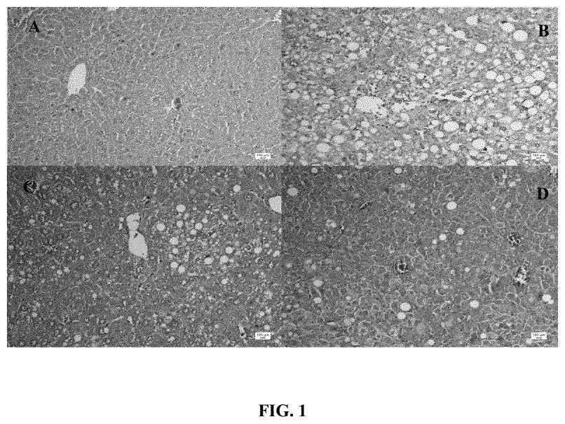

HE staining, under the microscope, it was seen that the liver tissue structure of the normal group was complete and clear, and the liver lobule structure was normal. The liver cells were arranged in hepatic cords and distributed radially around the central vein. The center of the cells had a large round nucleus, and the cytoplasm was uniform and no fat droplet. In the model group, liver steatosis was obvious. The liver cells were enlarged and contained large fat droplets. Some cells were found cell nuclei squeezed into the cell membrane, showing a large vesicular steatosis, and inflammatory cell infiltration was found. Punctate necrosis and focal necrosis were found in some cells, showing progressed to the stage of non-alcoholic steatohepatitis. Compared with the model group, the fat droplets in the Trezastilbenoside group were smaller and fewer, the pathological morphology of liver tissue was significantly improved, the fatty degeneration of vacuolated liver cells were significantly reduced, the infiltration of inflammatory cells was not obvious, and the cell arrangement was relatively neat and complete. These showed that Trezastilbenoside reduced the infiltration of hepatic inflammatory cells caused by high sugar and high fat, as shown in FIG. 1.

FIG. 1 is the pathological changes of mouse liver under the microscope of the present invention (HE, ×200); among them, A is the normal control group, B is the model group, C is the Trezastilbenoside low dose group, and D is the Trezastilbenoside high dose group.

It can be seen from the experimental results in FIG. 1 that, after 4 consecutive weeks of administration of Trezastilbenoside to model mice, the serum levels of TC, TG, and LDL were significantly reduced, and the HDL content was significantly increased, indicating that the drug has a lipid-regulating effect. The activities of AST and ALT in the serum were significantly weakened, indicating that the drug has the effect of improving liver function; the pathological morphology of the liver showed improvement in liver tissue steatosis and reduce of the infiltration of inflammatory factors, indicating that the drug has the effect of protecting the liver. It has good clinical value in the prevention and treatment of non-alcoholic fatty liver (NAFLD).

### Example 2

The tablet in this example was comprised of the following components: 10 g of Trezastilbenoside, 20 g of microcrystalline cellulose, 20 g of pregelatinized starch, 20 g of cross-linked polyvinylpyrrolidone, and 1 g of micropowder silica gel.

The above raw materials were mixed to obtain the Trezastilbenoside tablet in accordance with a conventional preparation method.

### Example 3

The capsule in this example was comprised of the following components: 10 g of Trezastilbenoside, 30 g of microcrystalline cellulose, 5 g of lactose, a proper amount of povidone K-30, and 1 g of magnesium stearate.

The above raw materials were mixed to obtain the Trezastilbenoside capsule in accordance with a conventional preparation method.

### Example 4

The granule in this example was comprised of the following components: 10 g of Trezastilbenoside, 20 g of mannitol, 20 g of lactose, 1 g of sodium cyclamate, 0.5 g of solid edible essence, and 1 g of xanthan gum.

The above raw materials were mixed to obtain the Trezastilbenoside granule in accordance with a conventional preparation method.

### Example 5

The dropping pill in this example was comprised of the following components: 5 g of Trezastilbenoside and 15 g of polyethylene glycol 6000.

The above raw materials were mixed to obtain the Trezastilbenoside dropping pill in accordance with a conventional preparation method.

The above are only the preferred examples of the present disclosure, and do not limit the present invention in any form. Although the present invention has been disclosed as the preferred examples, it is not intended to limit the present invention. Those skilled in the art familiar with this patent, can make slight changes or modification into equivalent embodiments with equivalent changes without departing from the scope of the technical solution of the present disclosure. For any simple modifications, equivalent changes and modifications made to the above embodiments based on the technology of the present invention without departing from the technical solution of the present invention, they still fall within the scope of the present invention.

1. A method of treating and/or preventing non-alcoholic fatty liver disease, comprising administering Trezastilbenoside to a subject in need thereof.

2. The method of claim 1, comprising administering Trezastilbenoside to the subject to improve the increase in body weight and/or liver weight of the subject.

3. The method of claim 1, comprising administering Trezastilbenoside to the subject to improve the increase in total cholesterol and/or triglycerides in serum of the subject.

4. The method of claim 1, comprising administering Trezastilbenoside to the subject to improve the increase in low density lipoprotein in serum of the subject.

5. The method of claim 1, comprising administering Trezastilbenoside to the subject to improve the increase in aspartate aminotransferase and/or alanine aminotransferase in serum of the subject.

6. The method of claim 1, comprising administering Trezastilbenoside to the subject to improve the decrease in high density lipoprotein in serum of the subject.

7. The method of claim 1, wherein the product includes a food product, a health product and a medicament.

8. The method of claim 1, wherein the product for treating and/or preventing non-alcoholic fatty liver disease is a medicament, comprising Trezastilbenoside and a pharmaceutically acceptable adjuvant.

9. The method of claim 8, wherein the medicament for treating and/or preventing non-alcoholic fatty liver disease is provided as various acceptable dosage forms;
preferably, the dosage forms include injections, tablets, capsules, powders, pills or oral solutions.

10. A product for treating and/or preventing non-alcoholic fatty liver disease, comprising Trezastilbenoside;
preferably, the product includes a food product, a health product and a medicament; and
preferably, the product is a medicament comprising Trezastilbenoside and a pharmaceutically acceptable adjuvant.
